(19) United States
(12) Patent Application Publication
(10) Pub. No.: US 2012/0309011 A1
(43) Pub. Date: Dec. 6, 2012

(54) TARGETING OF MODIFYING ENZYMES FOR PROTEIN EVOLUTION

(75) Inventors: Lei Wang, San Diego, CA (US); Jeffrey K. Takimoto, Rosemead, CA (US)

(73) Assignee: SALK INSTITUTE FOR BIOLOGICAL STUDIES, LA JOLLA, CA (US)

(21) Appl. No.: 13/505,540

(22) PCT Filed: Nov. 2, 2010
(86) PCT No.: PCT/US10/55161

§ 371 Date: Aug. 17, 2012

Related U.S. Application Data
(60) Provisional application No. 61/257,272, filed on Nov. 2, 2009.

(51) Int. Cl. C12P 21/00; C12Q 1/68; G01N 21/64; C12Q 1/02

(52) U.S. Cl. 435/6.12; 435/68.1; 435/29

(57) ABSTRACT

Methods and compositions for producing variants of a polypeptide are disclosed. Variants are generated using modifying enzymes specifically targeted to the polypeptide through the interaction of a T7 polymerase with a T7 polymerase promoter.

### CROSS-REFERENCE TO RELATED APPLICATION

This application claims the benefit of priority of U.S. provisional application Ser. No. 61/257,272, filed Nov. 2, 2009. The foregoing application is incorporated herein by reference in its entirety.

### FIELD OF THE INVENTION

The invention relates to the field of nucleic acid and protein modification. More particularly, the invention provides compositions and methods relating to the generation of mutations in nucleic acids and/or proteins.

### BACKGROUND OF THE INVENTION

Directed protein evolution is a very powerful tool to engineer proteins with new properties that are not found in natural proteins. To search protein sequences within weeks or months rather than millennia or millions of years for natural selection, large protein diversities need to be repetitively generated and screened very rapidly and efficiently. In vitro methods for creating genetic diversity are very powerful but laborious to apply repetitively when screening has to be done on transfected cells or organisms. The generation of protein variants in living cells would avoid repetitive transfection and reisolation of genes, but existing methods normally randomize the entire genome without focusing on the gene of interest.

Citation or identification of any document in this application is not an admission that such document is available as prior art to the present invention.

### SUMMARY OF THE INVENTION

The present invention solves the above and related problems in the art, by providing methods to engineer protein variants in living cells by targeting a modifying enzyme to the nucleic acid encoding the protein of interest and utilizing the specific interaction between a prokaryotic T7 polymerase and a T7 promoter. Using the methods provided herein, variants of a variety of polypeptides can be generated in a living cell and rapidly identified. Examples of modifying enzymes suitable for use in this invention include, but are not limited to, DNA modification enzymes, histone modification enzymes, transcription factor modification enzymes, and enzymes modifying ribonucleic acids and deoxyribonucleic acids. Without limitation, examples of proteins that can be used to generate variants thereof are fluorescent proteins, transcription factors, proteins involved in aminoacyl-tRNA synthesis, transporters, G-protein coupled receptors, and metabolic enzymes.

In certain embodiments, the invention provides a method for generating a variant of a target polypeptide, comprising introducing into a cell a target construct, said target construct comprising a nucleic acid comprising a T7 polymerase promoter operably linked to a nucleic acid encoding a target polypeptide, and a modifying construct, said modifying construct comprising a nucleic acid encoding a modifying enzyme linked to a T7 polymerase; expressing said modifying construct in said cell, thereby expressing said modifying enzyme linked to said T7 polymerase; recruiting said modifying enzyme linked to said T7 polymerase to said target construct through interaction of said T7 polymerase with said T7 polymerase promoter, and modifying said target polypeptide with said modifying enzyme, thereby generating a variant of said target polypeptide. In some embodiments, the cell is a eukaryotic cell. In other embodiments, the cell is a prokaryotic cell.

In certain embodiments, expressing said modifying construct further comprises stable expression in a mammalian cell. In some embodiments, said target construct comprises a nucleic acid comprising more than one copy of a T7 polymerase promoter operably linked to a nucleic acid encoding a target polypeptide. In certain embodiments, the T7 polymerase promoter further comprises a guanine at position −8. In some embodiments, the target construct further comprises an internal ribosome entry site (IRES).

In other embodiments, the target construct comprises an inducible promoter, and the method further comprises inducing a high level of expression of the target polypeptide, wherein the high level of expression of the target polypeptide is greater than corresponding rates of expression in the absence of said induction. In certain embodiments, the inducible promoter comprises a doxycyclin-dependent Tet-on promoter.

In some embodiments, said modifying construct further comprises a nuclear localization signal (NLS). In certain embodiments, said NLS is a SV40 NLS.

In some embodiments, said modifying enzyme is linked to the 5′-end of said T7 polymerase. In certain embodiments, said modifying construct comprises a nucleic acid encoding more than one copy of a modifying enzyme linked to a T7 polymerase. Suitable modifying enzymes include DNA editing enzymes, mRNA editing enzymes, and deaminases. Examples of suitable deaminases include an activation induced deaminase (AID) and an APOBEC protein. In certain further embodiments, said cell is capable of error-prone deoxyribonucleic acid repair.

In other embodiments, said modifying construct further comprises a nucleic acid encoding low-fidelity DNA repair proteins. Examples of low-fidelity DNA repair proteins include UNG1 and po1η.

In yet other embodiments, the method further comprises determining whether said cell exhibits a desired property. In certain embodiments, the method further comprises selecting said cell if said cell exhibits said desired property. In yet other embodiments, said exhibition of a desired property comprises expression of a polypeptide variant having a desired property. In some embodiments, said exhibition of a desired property comprises expression of a polypeptide variant having a desired property. In certain embodiments, the method further comprises isolating deoxyribonucleic acid (DNA) from said selected cell. In certain further embodiments, isolating DNA from said selected cell comprises amplification by polymerase chain reaction (PCR). In some embodiments, the method further comprises DNA sequencing. In other embodiments, said determining comprises determining a cell property using fluorescence activated cell sorting (FACS).

Examples of suitable modifying enzymes include DNA modifying enzymes, such as nucleases, recombinases, and methyltransferases; and protein modifying enzymes, such as histone modifying enzymes and transcription factor modifying enzymes, acetylases, kinases, methyltransferases, and ubiquitin ligases.

In yet other embodiments, the instant invention relates to a kit comprising a cell, a target construct comprising a nucleic acid comprising a T7 polymerase promoter operably linked to a nucleic acid encoding a target polypeptide, and a modifying construct comprising a nucleic acid encoding a modifying enzyme linked to a T7 polymerase. Examples of suitable cells for use in the kit include mammalian cells, bacterial cells, and yeast cells.

In certain embodiments, the kit comprises a target construct that further comprises a nucleic acid comprises more than one copy of a T7 polymerase promoter operably linked to a nucleic acid encoding a target polypeptide. In some embodiments, said T7 polymerase promoter further comprises a guanine at position −8. In other embodiments, said target construct further comprises an internal ribosome entry site. In yet other embodiments, said inducible promoter comprises a doxycyclin-dependent Tet-on promoter. In some embodiments, said modifying enzyme is fused N-terminal to said T7 polymerase. In other embodiments, said modifying construct further comprises a nucleic acid encoding more than one copy of a modifying enzyme linked to a T7 polymerase. In some embodiments, said modifying enzyme is an mRNA editing enzyme. In other embodiments, said modifying enzyme is a DNA modifying enzyme. In certain embodiments, said modifying enzyme is a histone modifying enzyme. In other embodiments, said modifying enzyme is a transcription factor modifying enzyme.

It is noted that in this disclosure and particularly in the claims, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

FIG. 1 depicts GFP translation in mammalian cells by the T7 RNA polymerase. A. Schematic of plasmids transfected into HEK293T cells. B. Cells transfected with T7-IRES-GFP only. C. Cells transfected with both T7-IRES-GFP and CMV-NLS-T7 RNAp.

FIG. 2 depicts AID orientation trials. A. Schematic of constructs to test AID location. AID was fused to T7 RNA polymerase in different ways to determine which one can provide a fusion protein that is functional in transcription and translation. B. FACS results of GFP expression from the reporter construct. pGTT7I-GFP represents T7-IRES-GFP. Other constructs are indicated by numbers as in A.

FIG. 3 depicts targeted AID test in E. coli. A. Targeted (T7 promoter) and non-targeted (Tac promoter) reporter construct to test AID induced deamination events. Kanamycin resistance cassette contains a L94P mutation to render the cells sensitive toward kanamycin B. Kanamycin resistance reversion assay. T7 represents targeted and Tac represents non-targeted reporter. T7 promoter targeted AID shows dramatically higher reversion frequency of L97P mutation than the non-targeted Tac promoter.

FIG. 4 depicts HEK293T stable cell line. Tet-on system to express mRFP1.2 at high levels in an inducible manner. The T7 promoter interacts with the T7 RNAp to target AID to mRFP1.2.

FIG. 5 depicts normalized fluorescent emission spectrum of mRFP1.2 and a mutant of mRFP1.2 after 20 rounds of fluorescence activated cell sorting. Spectrum was normalized to the maximum intensity. Difference in emission peaks was 10 nm.

FIG. 6a is a diagram depicting the activation of a B-cell by an antigen. The antigen binds to the variable region of the antibody bound to the b-cell. Mutations from somatic hypermutation are found specifically in the variable region.

FIG. 6b is a schematic showing that activation-induced deaminase deaminates cytosines in DNA to uracil.

FIG. 7a depicts activation-induced deaminase mediated mutations. Three different pathways can genomically inherit mutations when a cytosine is converted to a uracil in the genome: the replicative, UNG1, and the mismatch repair mechanism.

FIG. 7b is a schematic depicting one hypothesis where AID is either directly or indirectly interacting with an RNA polymerase to bind to its ssDNA substrate. Once targeted to the ssDNA by the RNA polymerase, the deaminase can then bind to the ssDNA that is exposed in the transcription bubble to cause deamination of cytosines to uracils.

FIG. 8a depicts an experimental diagram demonstrating the ability to target AID to a specific gene in E. coli. This is carried out by fusing AID to the T7 RNA polymerase. The T7 RNA polymerase targets the T7 promoter with high specificity and thereby brings the fused AID to the gene immediately downstream of the T7 promoter. To phenotypically demonstrate deamination of cytosines, a mutation (CCA) was inserted into the kanamycin resistance cassette to render the protein produce by the cassette inactive. However, if either of the cytosines is deaminated to a uracil and therefore through replicative mediated mutagenesis, the Pro94 will be changed to activate the kanamycin resistance enzyme, rendering cells bearing this deamination resistant toward kanamycin. Two different constructs were made, a targeted construct that used a T7 promoter and a non-targeted construct that used a prokaryotic promoter Tac.

FIG. 8b depicts an experimental procedure to demonstrate targeted deamination in E. coli. Plasmids bearing the deamination machinery and the reporter constructs are transformed into E. coli. The cells are grown in liquid culture while inducing for the expression of deamination machinery and the reporter construct. The cells are plated on kanamycin plates to identify the deamination events that are occurring on the reporter construct.

FIG. 9a depicts results from the kanamycin reversion assay in E. coli. Different constructs were tested to verify the targeting ability of the system. The boxes indicated the high reversion of kanamycin only when AID is fused to the T7 RNA polymerase and when the reporter construct contains the T7 promoter.

FIG. 9b is a chromatogram from sequencing results that shows that there is a population of plasmids within each cell that have deaminated one of the cytosines in the CCA mutation in the kanamycin resistance cassette.

FIG. 10a depicts an experimental procedure to mutate a fluorescent protein mCherry in BW310 cells. BW310 cells lack a uracil DNA glycosylase and therefore are unable to repair any cytosine to uracil conversions.

FIG. 10b is a table providing information on the fluorescent phenotype, mutations in the promoter, number of mutations, and the type of mutations caused by different deaminase constructs.

FIG. 11a depicts an example of the mutations that occurred in the mCherry fluorescent protein by Apobec-1 fused to T7 RNA polymerase.

FIG. 11b depicts removal of cytosine deamination hotspot in the T7 promoter. Apobec-1 was fused to a mutated T7 RNA polymerase (Q758C) that recognizes the new mutated T7 promoter. Table provides sequencing results on the mutated T7 promoter, the number of mutations in mCherry, the type of mutations, and the number of mutations per base pair that was sequenced.

FIG. 12a depicts an SV40 nuclear localization signal was attached to the T7 RNA polymerase to localize the protein to the nucleus. To demonstrate the activity of the polymerase, a T7 promoter upstream of a CMV internal ribosomal entry site (IRES) and GFP was co-transfected into cells.

FIG. 12b depicts fluorescent microscopy of cells that were transfected with the reporter or reporter and NLS-T7 RNA polymerase. Cells that were transfected with the reporter only resulted in no fluorescents while cells that were transfected with both the reporter and the NLS-T7 RNA polymerase had fluorescent cells present.

FIG. 13 depicts flow cytometry results of cells transfected with T7 RNA polymerase constructs and GFP reporter constructs. T7 RNA polymerase activity is demonstrated by its ability to express GFP after different deaminase genes were fused to the N-terminal end of the T7 RNA polymerase.

FIG. 14a depicts the different constructs that were made for E. coli and mammalian cells

FIG. 14b depicts the mutation rates that were found on cells that had the mammalian RFP construct integrated into the genome and transfected with Apobec1-T7 RNA polyermerase

FIG. 14c depicts retroviral infection constructs of the rtTA and targeted for mutagenesis mRFP1.2. Double infection creates a genomically integrated target gene (mRFP1.2), whose expression is inducibly controlled by the Tet-on system. A cocktail of constructs expressing different deaminases fused to the T7 RNA polymerase were transfected into the cell to mutate the target gene.

FIG. 15a depicts fluorescent-activated cell sorting strategy to shift the fluorescence emission peak through ratio sorting.

FIG. 15b depicts an example of ratio sorting and isolation of population of cells with shifted fluorescence emission.

FIG. 16a depicts fluorescent emission scan of cells from ratio sorting in different rounds.

FIG. 16b depicts fluorescence emission peak of ratio sorted cells.

FIG. 17 depicts sequencing results of the mRFP isolated from cells that were selected after 20 rounds of ratio sorting.

### DETAILED DESCRIPTION

The instant invention relates to a novel method for the specific introduction of mutations into a nucleic acid and/or protein. The gene-specific diversification techniques of the instant invention will provide new methods by which to selectively evolve proteins with novel properties to address a multitude of biological questions. For instance, the methods described herein can provide a new means to evolve aminoacyl-tRNA synthetases directly in mammalian cells for the incorporation of unnatural amino acids into proteins. Evolving mutant synthetases is currently only possible in bacteria and yeast.

By “target nucleic acid” or “target polypeptide” is meant a nucleic acid or polypeptide, respectively, that is to be modified. Any number of nucleic acids (e.g., DNA, RNA) or polypeptides may serve as the target nucleic acid or polypeptide. For example, the instant invention is suitable for conducting protein evolution of a protein of interest, such as for example, development of a fluorescent protein with a novel fluorescent capability, e.g., fluorescing at a different wavelength than a control fluorescent protein. A “target construct” comprises a target nucleic acid.

By “modifying” enzyme is meant any polypeptide capable of introducing a mutation into a target nucleic acid or polypeptide. A “modifying construct” comprises a nucleic acid encoding a modifying enzyme.

By “variant” with reference to a nucleic acid or polypeptide is meant any nucleic acid or polypeptide that is modified in some way.

A nucleic acid that is modified in accordance with a method of the invention is one that is mutated in any manner. For example, mutation of a target nucleic acid sequence according to the instant invention includes any substitution of, variation of, modification of, replacement of, deletion of or addition of one (or more) nucleotides from or to the sequence. A number of different types of modification to oligonucleotides are known in the art. These include methylphosphonate and phosphorothioate backbones. Where the polynucleotide is double-stranded, both strands of the duplex, either individually or in combination, are encompassed by the methods and compositions described herein. Where the polynucleotide is single-stranded, it is to be understood that the complementary sequence of that polynucleotide is also included.

A polypeptide that is modified in accordance with a method of the invention is one that is mutated in any manner. For example, mutation of a target amino acid sequence according to the instant invention includes any substitution of, variation of, modification of, replacement of, deletion of or addition of one (or more) amino acids from or to the sequence. Modification of amino acid sequences according to the invention also includes, without limitation, post-translational modifications, such as ubiquitination, methylation, acetylation, myristolation, glycosylation, truncation, lapidation and tyrosine, serine or threonine phosphorylation.

In certain embodiments, the invention provides a nucleic acid-modifying enzyme coupled to a T7 RNA polymerase, and a target nucleic acid coupled to a T7 promoter, wherein the nucleic acid-modifying enzyme is brought into close proximity with the target nucleic acid as a result of the interaction of the T7 RNA polymerase with the T7 promoter, such that the nucleic acid-modifying enzyme is able to modify the target nucleic acid.

In other embodiments, the modifying enzyme coupled to the T7 polymerase is a protein-modifying enzyme, such as a histone-modifying enzyme or a transcription factor-modifying enzyme. In embodiments where the modifying enzyme is a protein-modifying enzyme, the target protein is typically bound to nucleic acid near the T7 promoter or otherwise present in the cytosol or nucleosol near the T7 promoter. For example, the protein-modifying enzyme is brought into close proximity with a target polypeptide by means of the interaction of the T7 polymerase (with which the protein-modifying enzyme is coupled) with the T7 promoter. Typically, the target polypeptide is present at or near the T7 promoter, such that the protein-modifying enzyme is able to modify the target polypeptide at or near the T7 promoter as a result of its being brought into close proximity by means of the T7 polymerase interacting with the T7 promoter.

In other embodiments, the modifying enzyme fused to the T7 polymerase is a DNA repair enzyme, such as low-fidelity DNA repair enzymes uracil DNA glycosylase (UNG1) or polymerase η (Po1η).

In some embodiments, more than one modifying enzyme fused to a T7 polymerase is expressed in a host cell system of the invention. In certain embodiments, more than one modifying enzyme is fused to the T7 polymerase.

The modifying enzyme may be fused in any manner suitable to enhance its expression and/or activity in a host cell. For example, in certain embodiments, it will be preferable to fuse a modifying enzyme, such as AID, in tandem to a T7 polymerase. In certain embodiments, it may be desirable to fuse the modifying enzyme to the N-terminus of the T7 polymerase. In further embodiments, the modifying enzyme is fused in-frame with the polymerase. In yet other embodiments, the modifying enzyme is fused with one or more linker amino acids connecting the modifying enzyme and polymerase.

In other embodiments, for example, in embodiments where the host cell is a mammalian cell, the expression of the T7 polymerase may be facilitated by the addition of a nuclear localization signal, such as an SV40 nuclear localization signal.

Where expression of the target gene is desired, in certain embodiments, the target construct comprises a T7 promoter sequence, an internal ribosomal entry site (e.g., a CMV internal ribosomal entry site (IRES)), the target nucleic acid, and a T7 termination sequence. In certain embodiments, the target construct comprises more than one copy of the T7 promoter. In some embodiments, the T7 promoter is modified to enhance interaction with a binding partner (e.g., T7 RNA polymerase) and/or to enhance expression of the target gene. In certain further embodiments, the T7 promoter comprises a guanine at position −8.

In embodiments where it is desirable to increase the expression of the modifying enzyme/polymerase fusion protein and/or the target nucleic acid in mammalian cells, constitutive mammalian promoters and/or inducible promoters may be employed, such as, for example, a Tet-on system, as described herein. For instance, in certain embodiments, a Tet-on promoter is used that is a doxycyclin-dependent Tet-on promoter. Other conventional means in the art may be employed to increase the expression of a target nucleic acid and/or modifying enzyme/polymerase fusion protein. For example, in addition to the inducible Tet-on system, a constitutive promoter with high expression can be used. In mammalian cells, constitutive expression promoters that can be used include, but are not limited to, the CMV promoter, PGK promoter, SV40 promoter, β-actin promoter, and β-actin promoter coupled with CMV early enhance (CAGG).

In certain embodiments, the present invention relates to artificially targeting activation induced deaminase (AID) and homologs to mimic somatic hypermutation in non-B-cells. An in vivo method of gene specific diversification is provided herein, which in certain embodiments, employs human activation induced deaminase (AID) or apolipoprotein B mRNA editing enzyme, catalytic polypeptide-like (APOBEC) homologs. In doing so, the instant invention provides methods by which to identify proteins that are involved in the high mutation rate found in the variable region of the immunoglobulin loci in B-cells. The ability of AID to deaminate cytosine during somatic hypermutation (SHM), in combination with the DNA repair process, provides a mechanism by which mutations are generated within a gene without in vitro DNA manipulation techniques¹. In certain embodiments, AID is initially targeted to the gene of interest by fusing AID to an exogenous RNA polymerase. Without being bound to theory, Applicants hypothesized that by targeting AID through the RNA polymerase, AID will be in close proximity to single stranded DNA (ssDNA) created by the transcription bubble which will allow it to bind and deaminate the exposed cytosine base pairs². The deamination converts the cytosine to a uracil, allowing for mutagenesis of the gene through the DNA repair process³.

The methods of the present invention may be performed, by way of example, in vitro using transformed or non-transformed cells, immortalized cell lines, or in vivo using transformed animal models enabled herein.

Accordingly, the instant invention allows for nucleic acid and/or protein modification in any number of cell systems, including both prokaryotic and eukaryotic cell systems. Examples of suitable hosts for the nucleic acid and protein modification systems of the instant invention include organisms such as, without limitation, bacteria (e.g., E. coli), yeast (e.g., S. cerevisiae), plants (e.g., Arabidopsis thaliana), and worms (e.g., C. elegans), as well as single cell systems, such as, plant cells, insect cells, zebrafish, Xenopus, and mammalian cells, including, without limitation, mammalian cells from any number of mammalian cell lines, such as HEK293T and CHO cells. Examples of other suitable host cells include Hela, HEK293, HEK293A, ACHN, C6, Caco-2, COS, HCT-116, HepG2, HL60, HT29, HT-1080, HUVEC, IMR-90, Jurkat, K-562, LNCap, MCF-7, MSA-MB-231, MDA-MB-435, Molt-4, NCI-H460, NHFF, NIH-3T3, NTera2, PC-3, PC12, SK-BR3, SK-MEL-28, SK-OV-3, and THP-1. Animals included in the invention are any animals amenable to transformation techniques, including vertebrate and non-vertebrate animals and mammals.

The modifying enzyme may be any enzyme that is capable of introducing a modification into a nucleic acid and/or protein. In certain embodiments, the modifying enzyme is a nucleic acid-modifying enzyme. Examples of suitable nucleic acid-modifying enzymes include DNA editing enzymes and mRNA editing enzymes; deaminases, such as activation induced deaminase (AID) and APOBEC proteins; nucleases; recombinases; and methyltransferases; and homologs or derivatives thereof. In certain embodiments, the modifying enzyme is a peptide-modifying enzyme. Examples of suitable peptide-modifying enzymes include histone-modifying enzymes, acetylases, kinases, methyltransferases, ubiquitin ligases, SUMO ligases, demethylases, deacetylases, phosphotases, and homologs or derivatives thereof.

In certain embodiments, it will be desirable to modify a protein by introducing one or more mutations into the gene coding for the protein. In these embodiments, a modifying enzyme that is a nucleic acid-modifying enzyme is typically coupled with a T7 polymerase. The target gene to be modified will be operably linked to a corresponding T7 promoter. As a result of the interaction between the T7 polymerase and the T7 promoter, the nucleic acid-modifying enzyme will be brought into close proximity with the target gene, thereby enabling the modifying enzyme to mutate the gene accordingly.

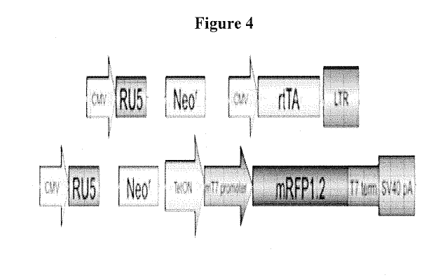

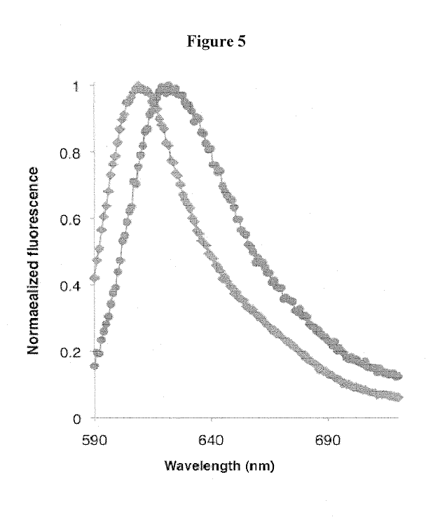

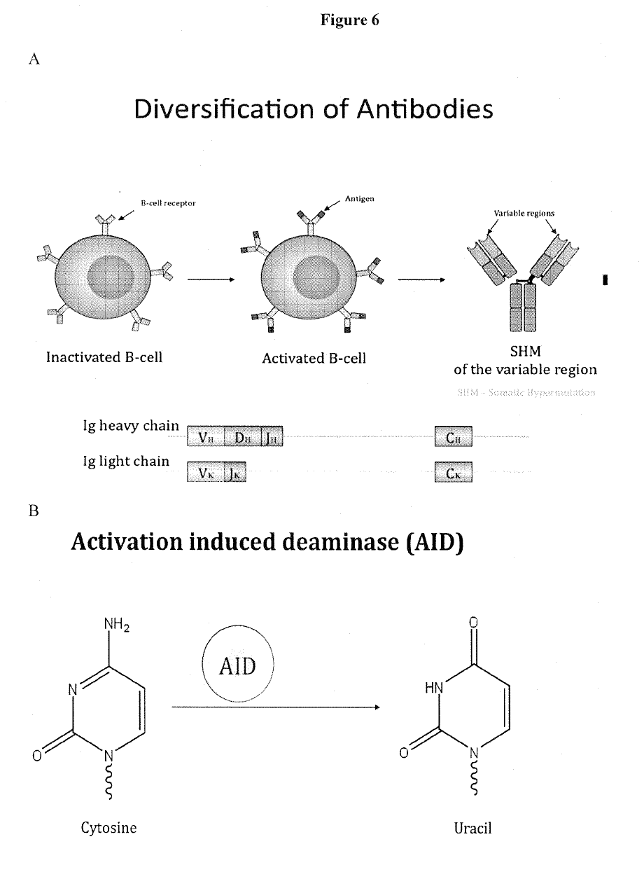

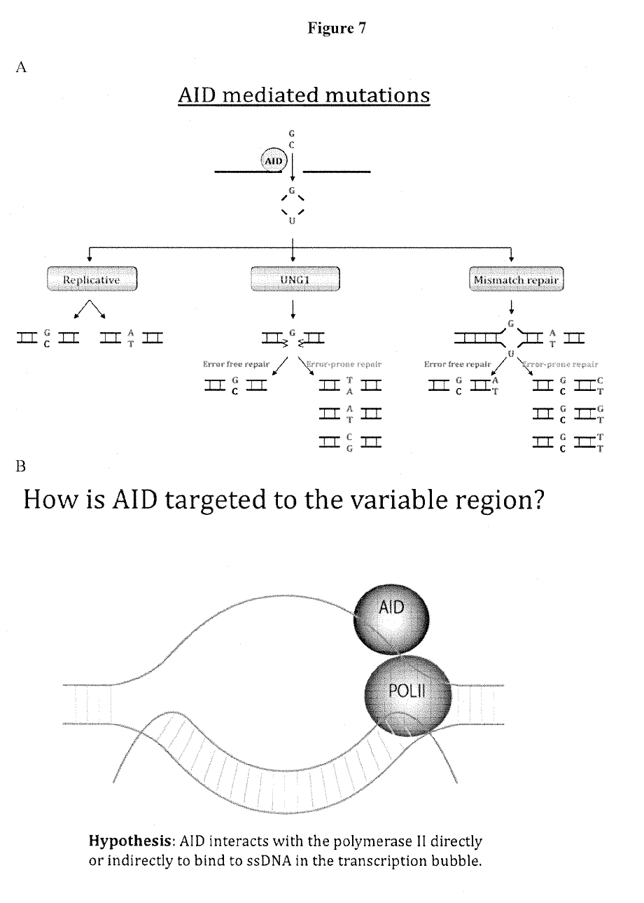

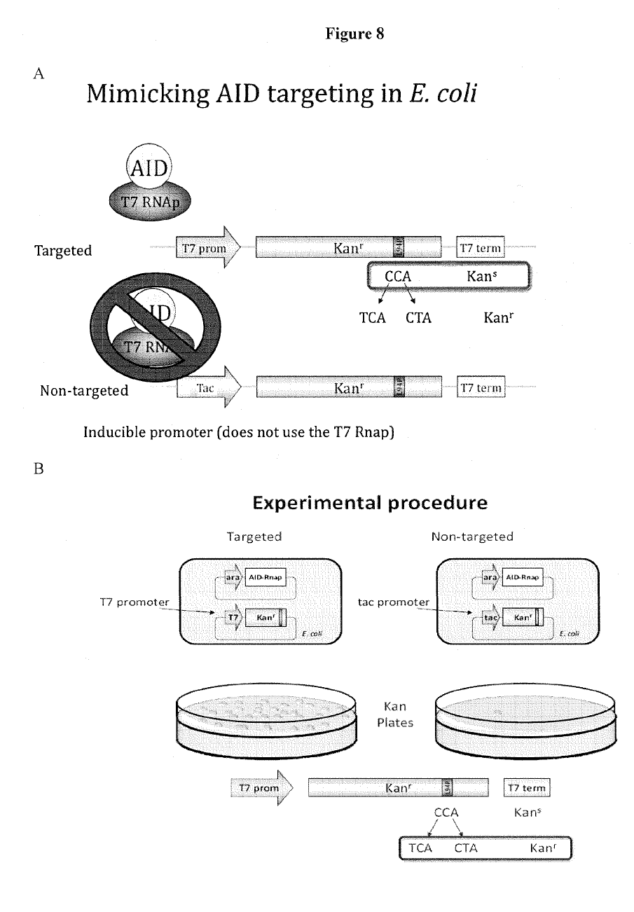

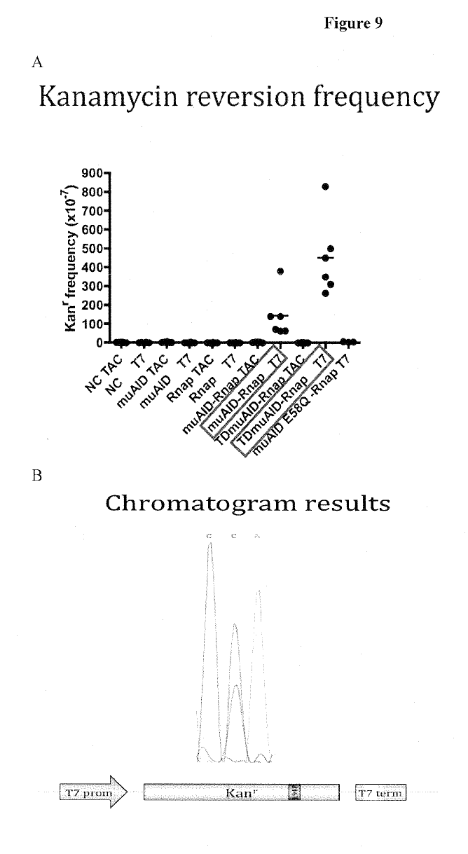

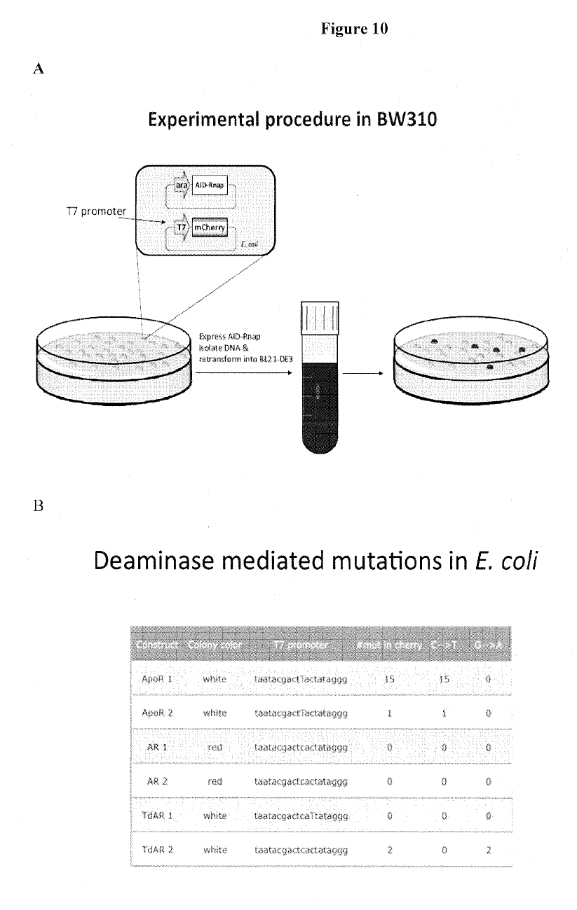

In some embodiments, the method directs AID and/or low-fidelity DNA repair proteins to a target nucleic acid, by employing a monoclonal cell line expressing GFP and rtTA. This can be created in a similar fashion as the mRFP1.2 stable cell line (FIG. 4). AID, uracil DNA glycosylase (UNG1), and polymerase (Po1η) can be fused to the N-terminal end of T7 RNAp to target GFP. When AID deaminates cytosine to a uracil, UNG recognizes uracil and removes the nucleotide. The removal of uracil creates an abasic site that is repaired by either a high or low-fidelity polymerase. Directing a low-fidelity polymerase, like Po1η, to the abasic site may increase the probability of repairing the site in a mutagenic fashion. Using the synthetic approach described herein of targeting proteins to a gene of interest, it may be possible to demonstrate that any gene can be targeted for mutation mimicking the variable region in the immunoglobulin loci in B-cells.

As used herein, the term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter code for amino acid residues are used herein.

The term “nucleic acid” encompasses DNA, RNA (e.g., mRNA, tRNA), heteroduplexes, and synthetic molecules capable of encoding a polypeptide and includes all analogs and backbone substitutes such as PNA that one of ordinary skill in the art would recognize as capable of substituting for naturally occurring nucleotides and backbones thereof. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence.

Unless otherwise indicated, nucleic acids are written left to right in 5′ to 3′ orientation; amino acid sequences are written left to right in amino to carboxy orientation, respectively.

As used herein, “hybridization” refers to the process by which one strand of nucleic acid base pairs with a complementary strand, as occurs during blot hybridization techniques and PCR techniques.

Hybridization conditions are based on the melting temperature (Tm) of the nucleic acid binding complex, as taught, e.g., in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of ordinary skill in the art, a maximum stringency hybridization can be used to identify or detect identical nucleotide sequences while an intermediate (or low) stringency hybridization can be used to identify or detect similar or related polynucleotide sequences.

In one aspect, the present invention employs nucleotide sequences that can hybridize to another nucleotide sequence under stringent conditions (e.g., 65° C. and 0.1×SSC {1×SSC=0.15 M NaCl, 0.015 M Na3 Citrate pH 7.0). Where the nucleotide sequence is double-stranded, both strands of the duplex, either individually or in combination, may be employed by the present invention. Where the nucleotide sequence is single-stranded, it is to be understood that the complementary sequence of that nucleotide sequence is also included within the scope of the present invention.

Stringency of hybridization refers to conditions under which polynucleic acid hybrids are stable. Such conditions are evident to those of ordinary skill in the field. As known to those of ordinary skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridization reaction is performed under conditions of higher stringency, followed by washes of varying stringency.

As used herein, high stringency includes conditions that permit hybridization of only those nucleic acid sequences that form stable hybrids in 1 M Na+ at 65-68° C. High stringency conditions can be provided, for example, by hybridization in an aqueous solution containing 6×SSC, 5×Denhardt's, 1% SDS (sodium dodecyl sulphate), 0.1 Na+ pyrophosphate and 0.1 mg/ml denatured salmon sperm DNA as non-specific competitor. Following hybridization, high stringency washing may be done in several steps, with a final wash (about 30 minutes) at the hybridization temperature in 0.2-0.1×SSC, 0.1% SDS.

It is understood that these conditions may be adapted and duplicated using a variety of buffers, e.g., formamide-based buffers, and temperatures. Denhardt's solution and SSC are well known to those of ordinary skill in the art as are other suitable hybridization buffers (see, e.g., Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridization conditions are typically determined empirically, as the length and the GC content of the hybridizing pair also play a role.

As used herein, a “synthetic” molecule is produced by in vitro chemical or enzymatic synthesis rather than by an organism.

The term “heterologous” with reference to a polynucleotide or protein refers to a polynucleotide or protein that does not naturally occur in a host cell.

As used herein, the term “expression” refers to the process by which a polypeptide is produced based on the nucleic acid sequence of a gene. The process includes both transcription and translation.

A “gene” refers to the DNA segment encoding a polypeptide.

By “homolog” is meant an entity having a certain degree of identity with the subject amino acid sequences and the subject nucleotide sequences. As used herein, the term “homolog” covers identity with respect to structure and/or function, for example, the expression product of the resultant nucleotide sequence has the enzymatic activity of a subject amino acid sequence. With respect to sequence identity, preferably there is at least 70%, 75%, 80%, 81%, 82%, 83%, 84%, 85%, 86%, 87%, 88%, 89%, 90%, 91%, 92%, 93%, 94%, 95%, 96%, 97%, 98%, or even 99% sequence identity. These terms also encompass allelic variations of the sequences. The term, homolog, may apply to the relationship between genes separated by the event of speciation or to the relationship between genes separated by the event of genetic duplication.

Relative sequence identity can be determined by commercially available computer programs that can calculate % identity between two or more sequences using any suitable algorithm for determining identity, using, for example, default parameters. A typical example of such a computer program is CLUSTAL. Advantageously, the BLAST algorithm is employed, with parameters set to default values. The BLAST algorithm is described in detail on the National Center for Biotechnology Information (NCBI) website.

The homologs of the peptides as provided herein typically have structural similarity with such peptides. A homolog of a polypeptide includes one or more conservative amino acid substitutions, which may be selected from the same or different members of the class to which the amino acid belongs.

In one embodiment, the sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The present invention also encompasses conservative substitution (substitution and replacement are both used herein to mean the interchange of an existing amino acid residue with an alternative residue) that may occur e.g., like-for-like substitution such as basic for basic, acidic for acidic, polar for polar, etc. Non-conservative substitution may also occur e.g., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Conservative substitutions that may be made are, for example, within the groups of basic amino acids (Arginine, Lysine and Histidine), acidic amino acids (glutamic acid and aspartic acid), aliphatic amino acids (Alanine, Valine, Leucine, Isoleucine), polar amino acids (Glutamine, Asparagine, Serine, Threonine), aromatic amino acids (Phenylalanine, Tryptophan and Tyrosine), hydroxylamino acids (Serine, Threonine), large amino acids (Phenylalanine and Tryptophan) and small amino acids (Glycine, Alanine).

Examples of homologs according to the invention include T7 RNA polymerase homologs, such as amino acids with at least 70%, at least 80%, at least 90%, at least 95%, at least 98% sequence identity to the amino acid sequence depicted in GenBank Accession No. NP_{—}041960.

Examples of homologs that may be employed in the methods of the instant invention also include AID homologs, such as nucleotides with at least 70%, at least 80%, at least 90%, at least 95%, at least 98% sequence identity to the nucleotide sequence depicted in GenBank Accession No. NG_{—}011588. Examples of homologs that may be employed in the methods of the instant invention also include APOBEC homologs, such as nucleotides with at least 70%, at least 80%, at least 90%, at least 95%, at least 98% sequence identity to the nucleotide sequence depicted in GenBank Accession No. NM_{—}012907 (APOBEC1), NM_{—}006789 (APOBEC2), NM_{—}021822 (APOBEC3G), and NM_{—}001193289 (APOBEC3A), or NM_{—}145699 (APOBEC3A).

Examples of homologs that may be employed in the methods of the instant invention also include Apob homologs, such as nucleotides with at least 70%, at least 80%, at least 90%, at least 95%, at least 98% sequence identity to the nucleotide sequence depicted in GenBank Accession No. NM_{—}019287 or NM_{—}009693.

Additional examples of suitable homologs that may be employed in the methods of the instant invention include vif homologs, such as nucleotides with at least 70%, at least 80%, at least 90%, at least 95%, at least 98% sequence identity to the nucleotide sequence depicted in Accession EU659813.

Further examples of suitable homologs that may be employed in the methods of the instant invention include UNG homologs, such as nucleotides with at least 70%, at least 80%, at least 90%, at least 95%, at least 98% sequence identity to the nucleotide sequence depicted in GenBank Accession No. NM_{—}003362 or NM_{—}080911.

Examples of suitable homologs that may be employed in the methods of the instant invention also include polymerase eta homologs, such as nucleotides with at least 70%, at least 80%, at least 90%, at least 95%, at least 98% sequence identity to the nucleotide sequence depicted in GenBank Accession No. NM_{—}006502.

Additional examples of suitable homologs that may be employed in the methods of the instant invention include endonuclease homologs, such as nucleotides with at least 70%, at least 80%, at least 90%, at least 95%, at least 98% sequence identity to the nucleotide sequence depicted in Accession ACY75846.

To aid in the detection of a protein or nucleic acid, labels can be used, such as any readily detectable reporter, for example, a fluorescent, bioluminescent, phosphorescent, radioactive, etc. reporter.

The present invention further contemplates direct and indirect labelling techniques. For example, direct labelling includes incorporating fluorescent dyes directly into a nucleotide sequence (e.g., dyes are incorporated into nucleotide sequence by enzymatic synthesis in the presence of labelled nucleotides or PCR primers). Direct labelling schemes include using families of fluorescent dyes with similar chemical structures and characteristics. In certain embodiments comprising direct labelling of nucleic acids, cyanine or alexa analogs are utilized. In other embodiments, indirect labelling schemes can be utilized, for example, involving one or more staining procedures and reagents that are used to label a protein in a protein complex (e.g., a fluorescent molecule that binds to an epitope on a protein in the complex, thereby providing a fluorescent signal by virtue of the conjugation of dye molecule to the epitope of the protein).

Embodiments of the invention also include methods of identifying mutated proteins and/or nucleic acids. For example, by comparing control cells with cells comprising the modifying construct and target construct of the present invention, the instant invention provides methods of identifying such mutated proteins and nucleic acids on the basis of their ability to provide a desired effect, for example, by affecting the expression of a target gene, the activity of a target protein, or other biochemical, histological, or physiological markers that distinguish cells bearing normal and mutated target gene or protein activity in control and transformed cells, respectively.

In accordance with another aspect of the invention, the mutated proteins and nucleic acids that are produced by the methods of the invention can be used as starting points for rational chemical design to provide ligands or other types of small chemical molecules.

DNA sequences encoding a modifying enzyme coupled to a T7 polymerase protein can be expressed in vitro by DNA transfer into a suitable host cell. “Host cells” are cells in which a vector can be propagated and its DNA expressed. The term also includes any progeny or graft material, for example, of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

The terms “recombinant expression vector” or “expression vector” refer to a plasmid, virus or other vehicle known in the art that has been manipulated by insertion or incorporation of a genetic sequence. Such expression vectors contain a promoter sequence which facilitates the efficient transcription of the inserted sequence. The expression vector typically contains an origin of replication, a promoter, as well as specific genes that allow phenotypic selection of the transformed cells.

Methods that are well known to those ordinarily skilled in the art can be used to construct expression vectors containing a modifying enzyme coding sequence linked to a T7 polymerase coding sequence and appropriate transcriptional/translational control signals or to construct expression vectors containing a target nucleic acid linked to a T7 promoter. These methods include in vitro recombinant DNA techniques, synthetic techniques, and in vivo recombination/genetic techniques.

A variety of host-expression vector systems may be utilized to express a coding sequence, such as nucleic acid sequence encoding a fusion protein comprising a modifying enzyme linked to a T7 polymerase, or a target protein operably linked to a T7 polymerase promoter. These include but are not limited to microorganisms such as bacteria transformed with recombinant bacteriophage DNA, plasmid DNA or cosmid DNA expression vectors containing a coding sequence; yeast transformed with recombinant yeast expression vectors containing a coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a coding sequence; insect cell systems infected with recombinant virus expression vectors (e.g., baculovirus) containing a coding sequence; or animal cell systems infected with recombinant virus expression vectors (e.g., retroviruses, adenovirus, vaccinia virus) containing a coding sequence, or transformed animal cell systems engineered for stable expression.

Depending on the host/vector system utilized, any of a number of suitable transcription and translation elements, including constitutive and inducible promoters, transcription enhancer elements, transcription terminators, etc. may be used in the expression vector (see e.g., Bitter et al. Methods in Enzymology 153, 516-544, 1987). For example, when cloning in bacterial systems, inducible promoters such as pL of bacteriophage 7, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used.

The term “operably linked” refers to functional linkage between a promoter sequence and a nucleic acid sequence regulated by the promoter. In certain embodiments, the operably linked promoter controls the expression of the nucleic acid sequence. Functional linkage between a promoter sequence and a nucleic acid sequence regulated by the promoter also includes embodiments where a nucleic acid sequence is modified by a modifying enzyme that is brought into close proximity to it by virtue of the interaction between the promoter sequence and a protein that interacts with the promoter sequence, for example, by virtue of the interaction between a T7 promoter and a T7 RNA polymerase linked to a modifying enzyme.

Accordingly, in some embodiments, the nucleic acid sequence need not be expressed, for example, in embodiments where the promoter serves to bring a DNA-modifying enzyme into close proximity to modify a target nucleic acid that is DNA, the target DNA need not be expressed to determine any mutations to the DNA but rather is instead isolated and sequenced to determine any mutations.

In other embodiments, for example, where a protein is the desired mutagenic target, (e.g., a DNA-binding protein such as a histone), the promoter (e.g., T7 promoter) serves to bind a protein that interacts with it (e.g., T7 polymerase fused to a modifying enzyme) and need not facilitate expression of the DNA-binding protein since the promoter principally serves only to bring the modifying enzyme into close enough proximity to be able to modify the target protein.

In other embodiments, the operably linked promoter controls the expression of the nucleic acid sequence. For example, in certain embodiments, the target nucleic acid that is mutated by the modifying enzyme fused to a promoter-binding protein and binding the promoter operably linked to the target nucleic acid sequence, is expressed under the control of the promoter and the resulting protein product is assayed for a desired activity (e.g., increased fluorescence).

It is also understood that the expression of structural genes may be driven by a number of promoters. Although the endogenous, or native promoter of a structural gene of interest may be utilized for transcriptional regulation of the gene, preferably, the promoter is a foreign regulatory sequence. For mammalian expression vectors, promoters capable of directing expression of the nucleic acid preferentially in a particular cell type may be used (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Camper and Tilghman, 1989. Genes Dev. 3: 537-546).

Promoters useful in the invention include both natural constitutive and inducible promoters as well as engineered promoters. Examples of inducible promoters useful in animals include those induced by chemical means, such as the yeast metallothionein promoter, which is activated by copper ions (Mett, et al. Proc. Natl. Acad. Sci., U.S.A. 90, 4567, 1993); and the GRE regulatory sequences which are induced by glucocorticoids (Schena, et al. Proc. Natl. Acad. Sci., U.S.A. 88, 10421, 1991). Other promoters, both constitutive and inducible will be known to those of ordinary skill in the art.

The instant invention is useful, among other things, as part of a strategy to create and identify proteins with a desired property. For example, the instant invention is useful for protein evolution, such as the development of proteins with improved properties, such as increased or decreased binding affinity, increased or decreased enzymatic activity, and/or improved agonist or antagonist capabilities.

Accordingly, the host cells of the instant invention may be chosen to facilitate determining whether a mutated protein exhibits a desired property. In certain embodiments, the cell will express the mutated polypeptide having the desired property. In certain further embodiments, the desired property is fluorescence, which can be detected by fluorescence activated cell sorting (FACS) or other suitable method known in the art.

In yet other embodiments, nucleic acid, e.g., DNA, is isolated from the host cell, amplified by polymerase chain reaction, and sequenced to determine what mutations were introduced into the target gene.

This invention further pertains to novel proteins and nucleic acids identified by the herein-described assays and uses thereof, for example, labeling proteins with improved characteristics, such as a red fluorescent protein that is brighter than or has a maximum emission peak greater than control red fluorescent proteins.

Also provided herein is a kit or package comprising a target construct comprising a nucleic acid encoding a T7 polymerase reporter operably linked to a nucleic acid encoding a target polypeptide, and a modifying construct comprising a nucleic acid encoding a modifying enzyme linked to a T7 polymerase, in packaged form, accompanied by instructions for use.

The invention will now be further described by way of the following non-limiting examples.

### Example 1

### Efficiently Target AID to a Specific Gene in HEK293 Cells to Reduce Possible Deleterious Genomic Mutations

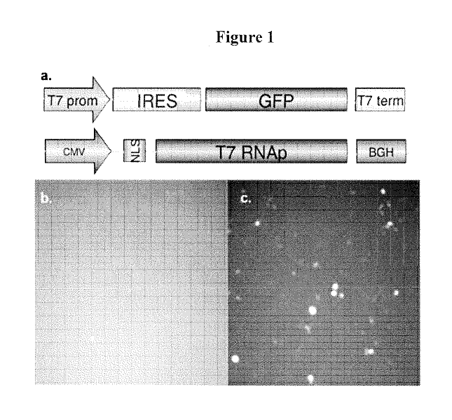

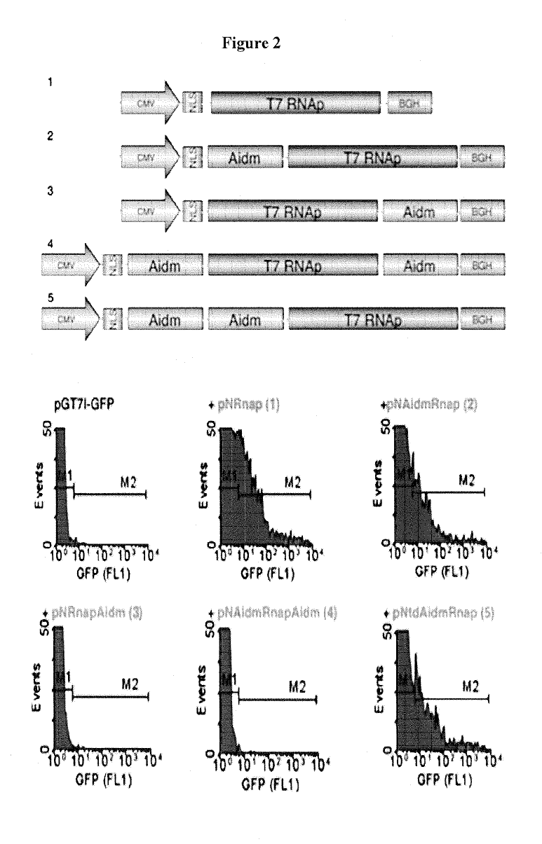

Initially, fusion of AID to the prokaryotic LexA protein was to be used as the artificial targeting mechanism. This fusion protein would create a local concentration of AID near the gene of interest to induce deamination and in turn mimic somatic hypermutation on a targeted gene. However, one major drawback to this plan stemmed from the inability of LexA to directly bind to the gene of interest⁴. LexA binds to its operator sequence and the coupled AID can reach and mutate sequences that are in close proximity to the operator sequence only. Sequences far away from the operator sequence will not be mutated. To circumvent this issue, Applicants fused AID to the T7 RNA polymerase (T7 RNAp). The prominent feature of the T7 RNA polymerase is its processivity, which enables it to travel along the target DNA and therefore reach sequences both immediate to and downstream of the T7 promoter. The T7 RNAp is widely used in protein expression in bacteria and in vitro transcription. Although not widely known, T7 RNAp can be used in mammalian cells with the addition of an SV40 nuclear localization signal⁵. Although T7 RNAp-driven transcription can occur in the nucleus with the addition of a nuclear localization signal, the RNA that is produced will not be translated due to the lack of a 5′ cap⁵. To demonstrate that a T7 RNAp can transcribe RNA in mammalian cells, a reporter construct was synthesized that contained a T7 promoter sequence, CMV internal ribosomal entry site (IRES), green fluorescent protein (GFP) gene, and a T7 termination sequence (FIG. 1a). The addition of the IRES allows for the translation of GFP RNA in the absence of the 5′ cap⁵. The reporter construct alone did not produce a high level of fluorescence, indicating the transcription and translation of GFP is absent or low (FIG. 1b). The co-transfection of the nuclear localized T7 RNAp and the reporter construct resulted in a clear production of GFP indicating that the nuclear-localized T7 RNAp is capable of transcribing RNA in mammalian cells (FIG. 1c). AID was fused to a T7 RNAp in different orientations to determine if AID fused to the 5′ or 3′ end of the T7 RNAp would render the polymerase inactive (FIG. 2a). When the reporter construct was co-transfected with the different AID orientations, FACS analysis revealed that only fusion proteins retaining transcriptional activity were those constructs in which AID was fused to the 5′ end of the T7 RNAp (FIG. 2b).

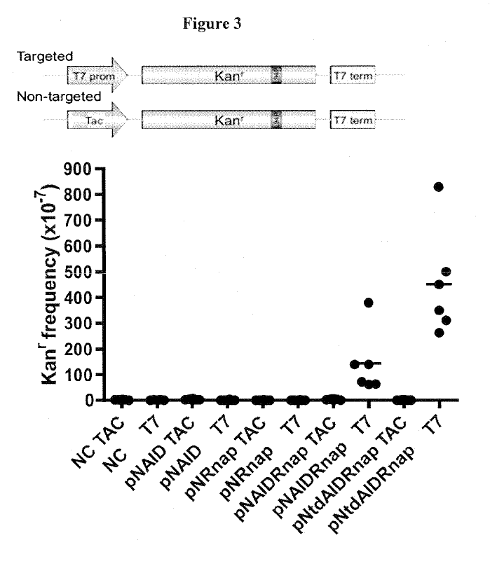

The ability of a T7 RNAp to recognize a promoter and transcribe without any other co-factors allows this system to be transferred to various organisms, including Escherichia coli, yeast, and mammalian cells. Therefore, the mutagenic activity of different AID constructs was verified in E. coli. In comparison to mammalian cells, E. coli has the benefit of fast growth and availability of selection markers. To demonstrate the gene specific deaminase activity in E. coli, a targeted and non-targeted reporter plasmid was constructed (FIG. 3a). The kanamycin resistance gene in the reporter construct contains a mutation corresponding to position 94 causing a leucine (TTG) to proline (CCA) amino acid change. This mutation makes the kanamycin resistance gene inactive and renders the E. coli sensitive to kanamycin⁶. However, if a cytosine in the proline codon is converted to a thymine, resistance to kanamycin is regained. The targeted reporter contains a T7 promoter allowing for interaction with the T7 RNAp-AID fusion, thus bringing AID into close proximity to the reporter ssDNA substrate in the transcription bubble. By targeting AID to the mutated kanamycin resistance gene, the mutagenic activity to the genome would be reduced. In comparison to AID alone, the targeted fused AID-RNAp provided a higher rate of reversion of the kanamycin resistance gene (FIG. 3b). One could argue that the higher rate of reversion is from the high transcription caused by the T7 RNAp; therefore, a non-targeted version of the construct that contains a lactose induce promoter (tac) was constructed. In this reporter, the endogenous machinery carried out the transcription of the kanamycin resistance gene to provide a non-targeted assessment of both free floating and the AID-T7 RNAp fusion protein. No major differences in reversion were seen, while the tac promoter was fully induced, in both the targeted and non-targeted form of AID (FIG. 3b). These results show that by fusing AID to the T7 RNAp, AID can be successfully targeted to the T7 promoter and in turn induce deamination to mimic somatic hypermutation in E. coli.

To test whether AID fused to the T7 RNAp can efficiently be targeted in mammalian cells, a stable cell line was created in human embryonic kidney cells (HEK) 293T that contain a tet-on system to express a red fluorescent protein (RFP) reporter (FIG. 4)⁵. The stable cell line has been created by a double infection of the RFP construct and the reverse tetracycline-controlled transactivator (rtTA). In this system, the main purpose of the nuclear-localized T7 RNAp is to target AID to the RFP gene, while the tet-on system expresses the protein. Although previously it was shown that the IRES in the RNA was sufficient for protein translation, integration of IRES-GFP construct into HEK293T cells resulted in low expression of the protein. It was concluded that the low copy number of the integrated reporter resulted in a reduced transcription level and in turn lowered translation of GFP. To circumvent this issue, the tet-on system provides the high transcription and translation of the protein while the T7 RNAp targets AID to induce deamination of the target gene. After AID targeting, the genomic DNA was purified and used as a template to amplify the RFP sequence. This amplified product was cloned into a bacterial plasmid and sequenced. The sequencing results demonstrated that mutations occurred on the RFP gene while no mutations were seen in the neomycin gene. The neomycin gene acts as a non-targeted control to verify the targeting ability of the T7 RNAp.

### Example 2

### Target Low Fidelity DNA Repair Machinery and AID to Synthetically Mimic the Mutation Rate of the Variable Regions of the Ig Loci in B-Cells

How AID is targeted to the variable region in the Ig loci has remained elusive. Recently, it was demonstrated that AID deaminates cytosine outside of the variable region. The majority of these deamination events are repaired in a non-mutagenic fashion. The high mutation rate at the variable region can be a result of the combination of AID targeting and low-fidelity repair. Another possibility is that AID is not targeted and deamination events are occurring throughout the genome. The deamination events are selectively repaired with high fidelity to reduce harmful mutations or with low fidelity as seen in somatic hypermutation. Using a synthetic approach based on the T7 RNAp targeting method, these questions can be addressed by targeting AID and proteins involved in low-fidelity DNA repair.

### Example 3

### Use of the Artificial Gene Specific Diversification Method to Evolve Proteins with Unique Properties

A potential problem with prolonged exposure to AID is the accumulation of deleterious mutations in the genome through non-specific deamination events. An additional benefit of using retroviruses to make a stable cell line is the ability to re-package the gene of interest back into a virus to infect fresh cells that do not contain deleterious mutations in the genome. Furthermore, the re-infection process can provide a step of recombination of the mutated genes by the innate mechanism of replication of the virus. Recombination allows for the enrichment of positive mutations to further increase the efficiency of evolving proteins²¹.

Using the previously mentioned stable cell line of HEK293T cells with an integrated T7 promoter-RFP, evolution experiments have been carried out to red shift the emission wavelength of RFP. mPlum was originally developed by evolving RFP in Ramos cells²². A major problem of the Ramos cell is that it mutates its genome in addition to the target exogenous gene that is integrated into the Ig loci. The Ramos cells therefore become sick and will die after some rounds of growth and selection. Those surviving cells might have a lower mutation ability, which is not desirable for the purpose of diversifying target genes. By focusing the mutation to the target gene and minimizing mutations elsewhere in the genome, Applicants T7 polymerase/promoter-based system greatly mitigates this problem. The retroviral strategy further ensures that mutants are safely transferred into fresh cells for additional rounds of mutation and evolution when necessary.

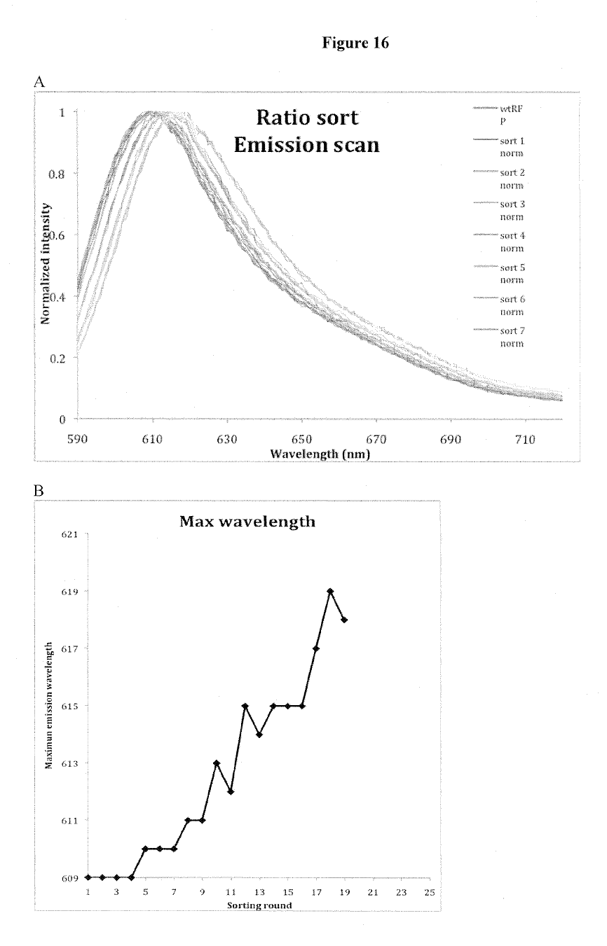

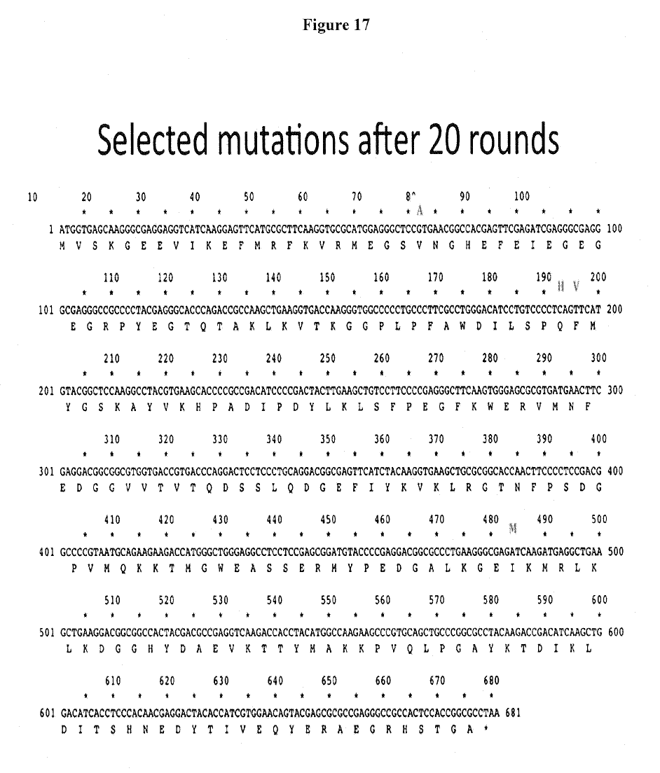

The RFP in the stable line was targeted for mutagenesis by the AID-T7 RNAp fusion protein and the evolution was monitored by fluorescent activated cell sorting (FACS). Using this method, Applicants have evolved mRFP1.2 to red shift approximately 10 nm after 20 rounds of sorting (FIG. 16). This result clearly indicates that Applicants' T7 RNA polymerase/promoter-based targeting mutation system works as designed. To further increase the mutation rate, a new construct for mutagenesis was developed. Using a sequence upstream of the T7 promoter that increases the promoter activity and using four repetitive elements, the targeting efficacy is increased and the mutation rate enhanced.

### Example 4

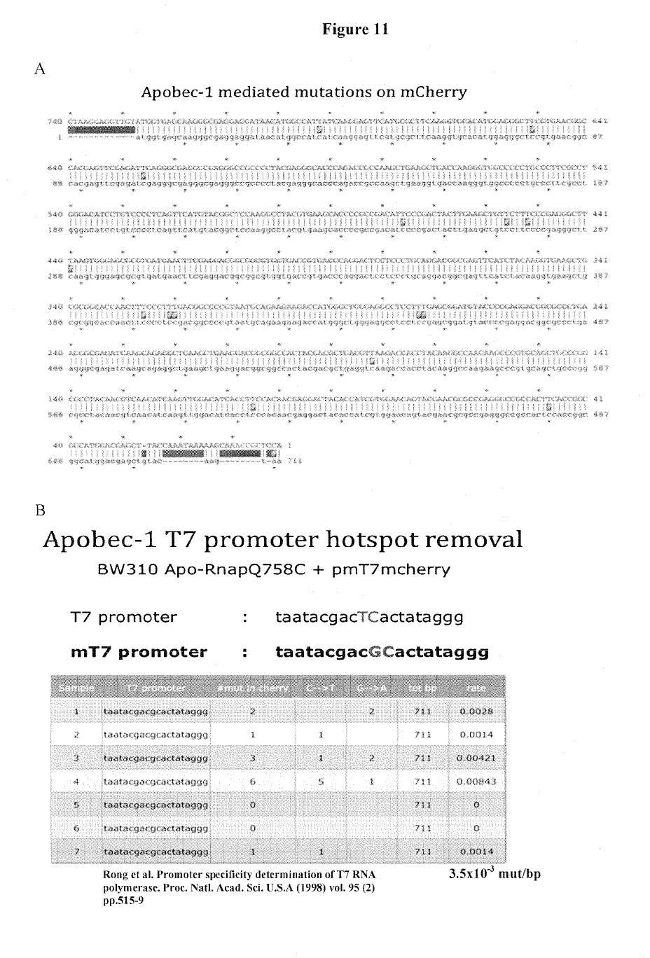

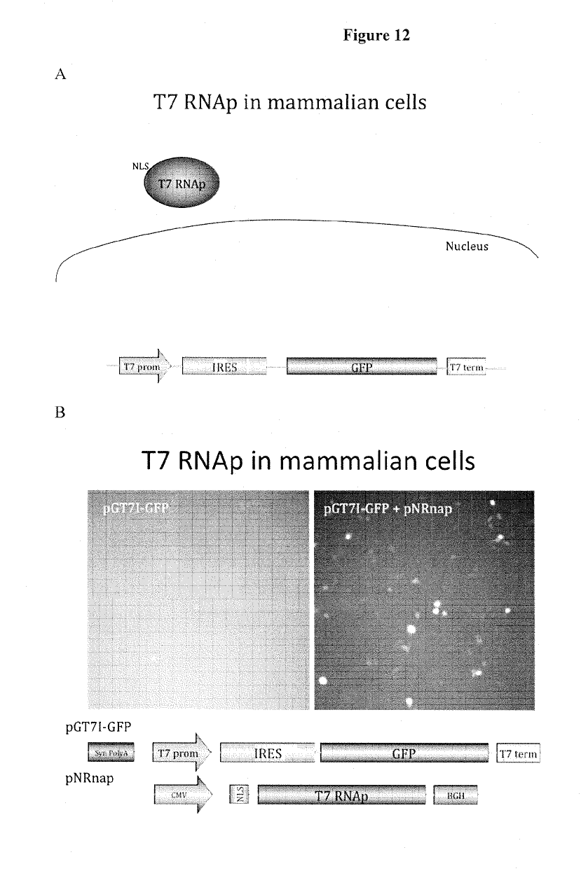

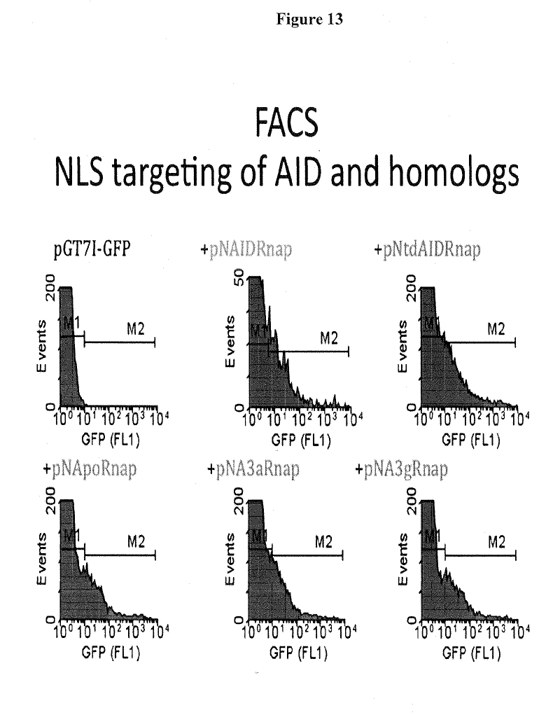

mCherry fluorescent protein was placed into the targeting construct that contains the T7 promoter and T7 terminator. Apobec-1 fused to the T7 RNA polymerase was expressed by the pARA promoter that is induced by arabinose. Apobec-1 was used as the deaminase in this system because of the high mutation rate that was previously observed. The two plasmids were transformed into the BW310 cell line. BW310 cells lack the uracil dna glycosylase protein that would normally excise uracil from DNA. The absence of this protein reduces the ability of the cell to correctly repair the deamination of cytosine to uracil. The fusion protein was expressed by the addition of 0.2% arabinose and grown overnight. The DNA was extracted from these cells and retransformed into new E. coli cells to isolate and amplify each plasmid separately. DNA is isolated from the newly transformed cells and sequenced. Results are presented in FIG. 11a.

Sequencing results indicated that mutations were occurring within the T7 promoter. The promoter encoded for a hotspot for mutations caused by deaminases. The hot spot was removed and a subsequent mutation was made into the T7 RNA polymerase to compensate for the mutation that was made into the promoter. The T7 RNA polymerase Q758C mutant polymerase can recognize the mutated promoter that lacks the hotspot for the deaminases to prevent the possibility of silencing the activity of the system by mutating the promoter used for targeting. Results are presented in FIG. 11b.

### Example 5

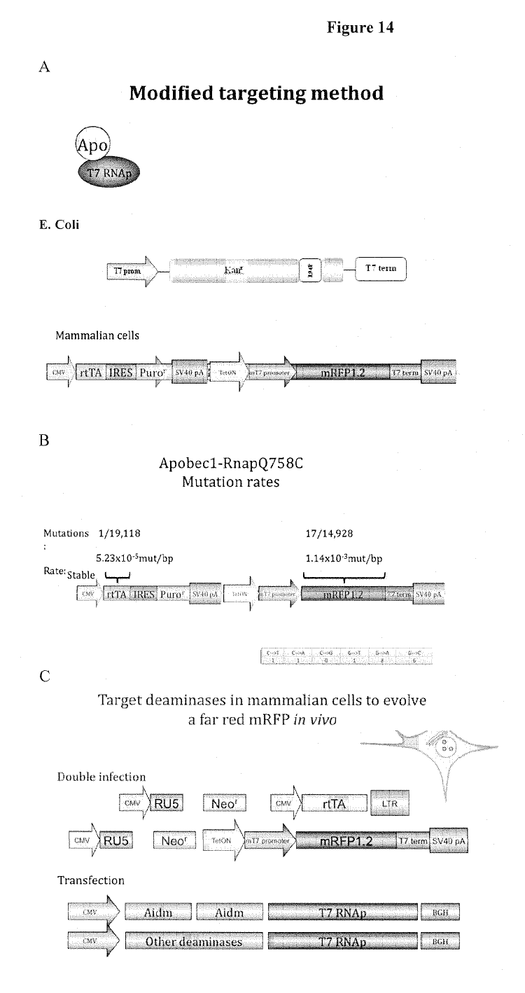

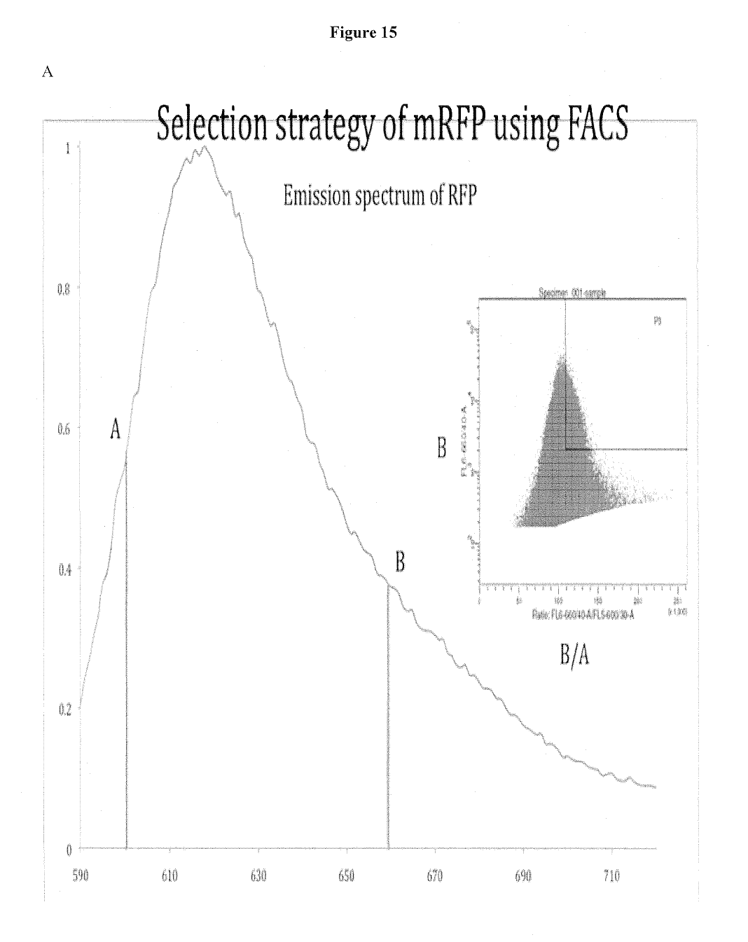

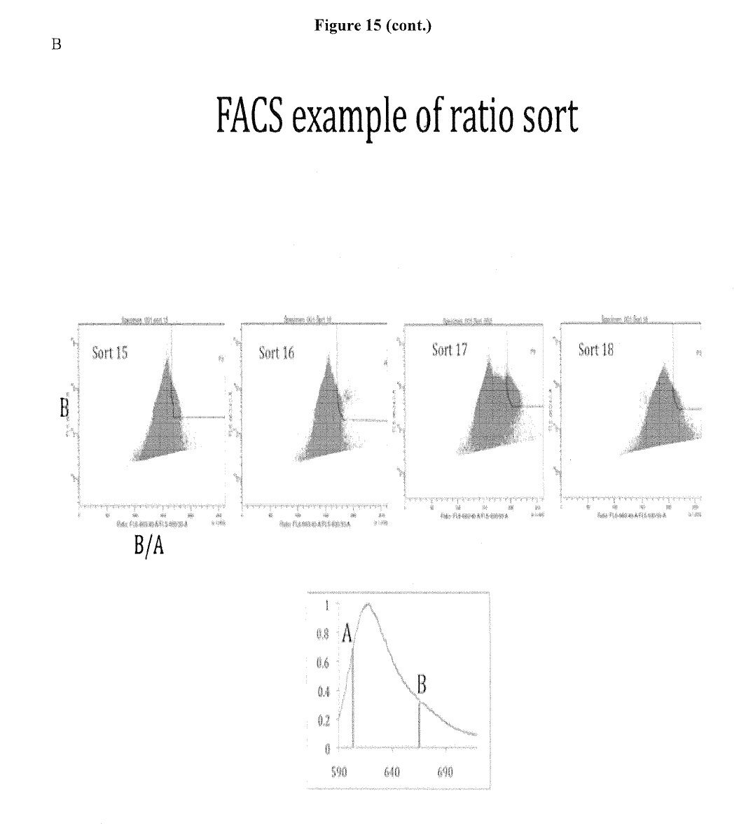

FIG. 14a provides a cartoon depiction of a targeting system according to the invention in mammalian cells. A nuclear-localized deaminase is fused to the mutated T7 RNA polymerase Qt58C and is targeted to the mRFP1.2 in the genome. The RFP1.2 was placed into the genome through the selection of the puromycin resistance cassette that was placed into the construct. The construct also contains the Tet-on system for the high expression of mRFP1.2. The Tet-On system is for the expression of the fluorescent protein, while the mutated T7 promoter is for targeting the T7 RNA polymerase to the gene of interest.

After a population stable cell line expressing the mRFP1.2 for targeted mutagenesis was created, the deaminase fused to the RNA polymerase was transfected into these cells. The genomic DNA from these cells was extracted and regions that are non-targeted and targeted were amplified and sequenced to compare the mutational spectrum. See FIG. 14b.

A different system was used to create a stable cell line that contains the targeting machinery. Instead of creating a stable cell line through plasmid integration, this system utilizes the MMLV retroviral infection system. The target is integrated through infection, and the deaminase fused to the T7 RNA polymerase Q758C is transfected into the cell to induce targeted mutagenesis. See FIG. 14c.

### Materials and Methods

### Bacterial Assay

Kanamycin reversion assays were carried out according to reference²³.

### Cell Culture and Transfection

A clonal HeLa GFP-TAG reporter stable cell line³ and HEK293T cells were cultured in Dulbecco's modified Eagle's medium (DMEM, Mediatech) supplemented with 10% fetal bovine serum (FBS, Mediatech). Cells were transfected with plasmid DNA using Lipofectamine 2000 according to the protocol of the vendor (Invitrogen).

### Ratio Sorting

Ratio sorting was carried out according to reference²².

### Retroviral Infection

All retroviral infections were carried out according to reference²².

### Flow Cytometry

Flow cytometry and fluorescence imaging were carried out according to reference.²²

### REFERENCES

1. Di Noia, J. M. & Neuberger, M. S. Molecular Mechanisms of Antibody Somatic Hypermutation. Annu Rev Biochem (2007).
2. Odegard, V. H. & Schatz, D. G. Targeting of somatic hypermutation. Nature reviews. Immunology 6, 573-83 (2006).
3. Vallur, A. C., Yabuki, M., Larson, E. D. & Maizels, N. AID in antibody perfection. Cellular and molecular life sciences: CMLS 64, 555-65 (2007).
4. Smith, G. M. et al. The Escherichia coli LexA repressor-operator system works in mammalian cells. The EMBO journal 7, 3975-82 (1988).
5. Meyer-Ficca, M. L. et al. Comparative analysis of inducible expression systems in transient transfection studies. Analytical biochemistry 334, 9-19 (2004).
6. Ramiro, A., Stavropoulos, P., Jankovic, M. & Nussenzweig, M. Transcription enhances AID-mediated cytidine deamination by exposing single-stranded DNA on the nontemplate strand. Nature Immunology 4, 452-6 (2003).
7. Wang, L., Brock, A., Herberich, B. & Schultz, P. G. Expanding the genetic code of Escherichia coli. Science (New York, N.Y.) 292, 498-500 (2001).
8. Wang, L., Xie, J. & Schultz, P. G. Expanding the genetic code. Annual review of biophysics and biomolecular structure 35, 225-49 (2006).
9. Wang, Q. & Wang, L. New methods enabling efficient incorporation of unnatural amino acids in yeast. Journal of the American Chemical Society 130, 6066-7 (2008).
10. Chen, S., Schultz, P. G. & Brock, A. An improved system for the generation and analysis of mutant proteins containing unnatural amino acids in Saccharomyces cerevisiae. Journal of molecular biology 371, 112-22 (2007).
11. Liu, W., Brock, A., Chen, S. & Schultz, P. G. Genetic incorporation of unnatural amino acids into proteins in mammalian cells. Nature methods 4, 239-44 (2007).
12. Wang, W. et al. Genetically encoding unnatural amino acids for cellular and neuronal studies. Nat Neurosci (2007).
13. Ibba, M. & Soll, D. Aminoacyl-tRNA synthesis. Annu Rev Biochem 69, 617-50 (2000).
14. Kobayashi, T. et al. Structural snapshots of the KMSKS loop rearrangement for amino acid activation by bacterial tyrosyl-tRNA synthetase. Journal of molecular biology 346, 105-17 (2005).
15. Yaremchuk, A., Kriklivyi, I., Tukalo, M. & Cusack, S. Class I tyrosyl-tRNA synthetase has a class II mode of cognate tRNA recognition. The EMBO journal 21, 3829-40 (2002).
16. Krissinel, E. & Henrick, K. Secondary-structure matching (SSM), a new tool for fast protein structure alignment in three dimensions. Acta crystallographica Section D, Biological crystallography 60, 2256-68 (2004).
17. Deiters, A. et al. Adding amino acids with novel reactivity to the genetic code of Saccharomyces cerevisiae. Journal of the American Chemical Society 125, 11782-3 (2003).
18. Chin, J. W. et al. An expanded eukaryotic genetic code. Science (New York, N.Y.) 301, 964-7 (2003).
19. Malandro, M. S. & Kilberg, M. S. Molecular biology of mammalian amino acid transporters. Annu Rev Biochem 65, 305-36 (1996).
20. Tsien, R. Y. A non-disruptive technique for loading calcium buffers and indicators into cells. Nature 290, 527-8 (1981).
21. Crameri, A., Raillard, S. A., Bermudez, E. & Stemmer, W. P. DNA shuffling of a family of genes from diverse species accelerates directed evolution. Nature 391, 288-91 (1998).
22. Wang, L., Jackson, W. C., Steinbach, P. A. & Tsien, R. Y. Evolution of new nonantibody proteins via iterative somatic hypermutation. Proceedings of the National Academy of Sciences of the United States of America 101, 16745-9 (2004).
23. Besmer, E., Market, E. & Papavasiliou, F. N. The transcription elongation complex directs activation-induced cytidine deaminase-mediated DNA deamination. Molecular and cellular biology 26, 4378-85 (2006).
24. Thomas, P. & Smart, T. G. HEK293 cell line: a vehicle for the expression of recombinant proteins. Journal of pharmacological and toxicological methods 51, 187-200 (2005).

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

1. A method for generating a variant of a target polypeptide, comprising:
introducing into a cell a target construct, said target construct comprising a nucleic acid comprising a T7 polymerase promoter operably linked to a nucleic acid encoding a target polypeptide, and a modifying construct, said modifying construct comprising a nucleic acid encoding a modifying enzyme linked to a T7 polymerase;
expressing said modifying construct in said cell, thereby expressing said modifying enzyme linked to said T7 polymerase;
recruiting said modifying enzyme linked to said T7 polymerase to said target construct through interaction of said T7 polymerase with said T7 polymerase promoter, and
modifying said target polypeptide with said modifying enzyme, thereby generating a variant of said target polypeptide.

2. The method of claim 1, wherein said cell is a eukaryotic cell.

3. The method of claim 1, wherein said cell is a prokaryotic cell.

4. The method of claim 1, wherein said expressing said modifying construct further comprises stable expression in a mammalian cell.

5. The method of claim 1, wherein said target construct comprises a nucleic acid comprising more than one copy of a T7 polymerase promoter operably linked to a nucleic acid encoding a target polypeptide.

6. The method of claim 1, wherein said T7 polymerase promoter further comprises a guanine at position −8.

7. The method of claim 1, wherein said target construct further comprises an internal ribosome entry site (IRES).

8. The method of claim 1, wherein said target construct comprises an inducible promoter, the method further comprising inducing a high level of expression of said target polypeptide, wherein said high level of expression of said target polypeptide is greater than corresponding rates of expression in the absence of said induction.

9. The method of claim 8, wherein said inducible promoter comprises a doxycyclin-dependent Tet-on promoter.

10. The method of claim 1, wherein said modifying construct further comprises a nuclear localization signal (NLS).

11. The method of claim 10, wherein said NLS is an SV40 NLS.

12. The method of claim 1, wherein said modifying enzyme is linked to the 5′-end of said T7 polymerase.

13. The method of claim 1, wherein said modifying construct further comprises a nucleic acid encoding more than one copy of a modifying enzyme linked to a T7 polymerase.

14. The method of claim 1, wherein said modifying enzyme is a DNA editing enzyme.

15. The method of claim 1, wherein said modifying enzyme is an mRNA editing enzyme.

16. The method of claim 1, wherein said modifying enzyme is a deaminase.

17. The method of claim 16, wherein said deaminase is an activation induced deaminase (AID).

18. The method of claim 16, wherein said deaminase is an APOBEC protein.

19. The method of claim 14, wherein said cell is capable of error prone deoxyribonucleic acid repair.

20. The method of claim 1, wherein said modifying construct comprises a nucleic acid encoding one or more low-fidelity DNA repair proteins.

21. The method of claim 20, wherein said low-fidelity DNA repair proteins are UNGI and po1η.

22. The method of claim 1, further comprising determining whether said cell exhibits a desired property.

23. The method of claim 22, further comprising selecting said cell if said cell exhibits said desired property.

24. The method of claim 22, wherein said exhibition of a desired property comprises expression of a polypeptide variant having a desired property.

25. The method of claim 23, wherein said exhibition of a desired property comprises expression of a polypeptide variant having a desired property.

26. The method of claim 23, further comprising isolating deoxyribonucleic acid (DNA) from said selected cell.

27. The method of claim 26, wherein said isolating DNA from said selected cell comprises amplification by polymerase chain reaction (PCR).

28. The method of claim 27, further comprising DNA sequencing.

29. The method of claim 22, wherein said determining comprises determining a cell property using fluorescence activated cell sorting (FACS).

30. The method of claim 1, wherein said modifying enzyme is DNA modifying enzyme.

31. The method of claim 30, wherein said DNA modifying enzyme is a nuclease.

32. The method of claim 30, wherein said DNA modifying enzyme is a recombinase.

33. The method of claim 30, wherein said DNA modifying enzyme is a methyltransferase.

34. The method of claim 1, wherein said modifying enzyme is a protein modifying enzyme.

35. The method of claim 34, wherein said protein modifying enzyme is a histone modifying enzyme.

36. The method of claim 34, wherein said protein modifying enzyme is transcription factor modifying enzyme.

37. The method of claim 34, wherein said protein modifying enzyme is a methyltransferase.

38. The method of claim 34, wherein said protein modifying enzyme is a ubiquitin ligase, acetylase, or kinase.

39. A kit comprising a cell, a target construct comprising a nucleic acid comprising a T7 polymerase promoter operably linked to a nucleic acid encoding a target polypeptide, and
a modifying construct comprising a nucleic acid encoding a modifying enzyme linked to a T7 polymerase.

40. The kit of claim 39, wherein said cell is a mammalian cell.

41. The kit of claim 39, wherein said cell is a bacteria cell.

42. The kit of claim 39, wherein said cell is a yeast cell.

43. The kit of claim 39, wherein said target construct comprises a nucleic acid comprising more than one copy of a T7 polymerase promoter operably linked to a nucleic acid encoding a target polypeptide.

44. The kit of claim 39, wherein said T7 polymerase promoter further comprises a guanine at position −8.

45. The kit of claim 39, wherein said target construct further comprises an internal
ribosome entry site.

46. The kit of claim 39, wherein said target construct comprises an inducible promoter that is a doxycyclin-dependent Tet-on promoter.

47. The kit of claim 39, wherein said modifying enzyme is fused N-terminal to said T7 polymerase.

48. The kit of claim 39, wherein said modifying construct comprises a nucleic acid encoding more than one copy of a modifying enzyme linked to a T7 polymerase.

49. The kit of claim 39, wherein said modifying enzyme is an mRNA editing enzyme.

50. The kit of claim 39, wherein said modifying enzyme is a DNA modifying enzyme.

51. The kit of claim 39, wherein said modifying enzyme is a histone modifying enzyme.

52. The kit of claim 39, wherein said modifying enzyme is a transcription factor modifying enzyme.

53. The method of claim 15, wherein said cell is capable of error prone deoxyribonucleic acid repair.

54. The method of claim 16, wherein said cell is capable of error prone deoxyribonucleic acid repair.

55. The method of claim 17, wherein said cell is capable of error prone deoxyribonucleic acid repair.

56. The method of claim 18, wherein said cell is capable of error prone deoxyribonucleic acid repair.


SEQUENCE LISTING (4 sequences)

SEQ ID NO: 1 - DNA, 740 bp, Artificial Sequence
  Synthetic polynucleotide sequence
ctaaggaccttgtatcctcaccaagggcgaggaggataacatggccattatcaaggaggtcattcccttcaaggtgcacatggagggcttcgtgaacggccacgagttcgagattgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacattcccgactacttgaagctgttctttcccgagggctttaagtgggagcgcgtgatgaacttcgaggacggcggcgtggtgaccgtgacccaggactcctccctgcaggacggcgagttcacctacaaggtgaagctgcgcggcaccaactttccctttgacggccccgtaatgcagaagaagaccatgggctgggaggcctcctttgagcggatgtaccccgaggacggcgccctgaagggcgagatcaagcagaggctgaagctgaaggacggcggccactacgacgctgaggttaagaccacctacaaggccaagaagcccgtgcagctgcccggcgcctacaacgtcaacatcaagttggacatcaccttccacaacgaggactacaccatcgtggaacagtacgaacgcgccgagggccgccaactcaccggcggcatggacgagcttaccaaataaaaagcaaaccgctcca

SEQ ID NO: 2 - DNA, 708 bp, Artificial Sequence
  Synthetic polynucleotide sequence
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcgtggtgaccgtgacccaggactcctccctgcaggacggcgagttcatctacaaggtgaagctgcggggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 3 - DNA, 681 bp, Artificial Sequence
  Synthetic polynucleotide sequence
atggtgagcaagggcgaggaggtcatcaaggagttcatgcgcttcaaggtgcgcatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgcctaa

SEQ ID NO: 4 - protein, 226 aa, Artificial Sequence
  Synthetic polypeptide sequence
MVSKGEEVIKEFMRFKVRMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYMAKKPVQLPGAYKTDIKLDITSHNEDYTIVEQYERAEGRHSTGA